(19) United States
(12) Patent Application Publication
(10) Pub. No.: US 2009/0216177 A1
(43) Pub. Date: Aug. 27, 2009

(54) CATHETER-TYPE IONTOPHORESIS DEVICE

(75) Inventors: Hidero Akiyama, Tokyo (JP); Hiroyoshi Kawakami, Tokyo (JP); Mizuo Nakayama, Tokyo (JP); Takehiko Matsumura, Tokyo (JP); Akihiko Matsumura, Tokyo (JP)

Correspondence Address: SEED INTELLECTUAL PROPERTY LAW GROUP PLLC, 701 FIFTH AVE, SUITE 5400, SEATTLE, WA, 98104, US

(73) Assignee: TTI ellebeau,Inc, Tokyo (JP)

(21) Appl. No.: 12/066,370

(22) PCT Filed: Sep. 14, 2006
(86) PCT No.: PCT/JP2006/318239

§ 371 Date: May 11, 2009

(30) Foreign Application Priority Data
Sep. 16, 2005 (JP) 2005-270862

(51) Int. Cl. A61N 1/30; A61N 5/06

(52) U.S. Cl. 604/21

(57) ABSTRACT

A catheter-type iontophoresis device is adapted to cause a drug solution to permeate into a cancer site or the like of a digestive organ on a pinpoint basis by iontophoresis.

The catheter-type iontophoresis device includes a working electrode assembly and a non-working electrode assembly for administering a drug by iontophoresis. A DC electric power source is connected to the working electrode assembly and the non-working electrode assembly with opposite polarities, and a rod-like member supports the working electrode assembly and the non-working electrode assembly at its tip. The rod-like member is detachably supported at a tip of a flexible cable supported by an endoscopic device.

### CROSS-REFERENCE TO RELATED APPLICATIONS

This application is the U.S. national stage of international application no. PCT/JP2006/318239, filed Sep. 14, 2006, which claims benefit from Japanese application no. 2005-270862, filed Sep. 16, 2005, which applications are hereby incorporated by reference in their entirety.

### BACKGROUND

1. Technical Field

This description relates to an iontophoresis device for administering a drug to an organism.

2. Description of the Related Art

Iontophoresis devices may be used to administer a drug solution through a biological interface (e.g., skin or a mucosa). The contact area with the skin or mucosa is often a relatively large area of at least about 20 mm in diameter.

In other medical applications, direct injection may be used to increase a drug's therapeutic effect. For example, an injection may be made at: a region targeted for endoscopic surgery; a mucosa in a nasal cavity; a mucosa in an oral cavity; an esophageal region; a stomach; a small intestine; a large intestine; an anal region; an affected area prior to a laparoscopic operation for lung cancer therapy; part of an organism exposed in a laparotomy, etc.

Drug delivery by iontophoresis rather than by injection is non-invasive and often preferable.

In a treatment called photodynamic therapy (PDT), after a photosensitive substance has been administered, light may be applied to carry out an anti-cancer reaction. However, a patient must not be exposed to sunlight during such treatment because the photosensitive substance circulates throughout his or her body. In addition, the photosensitive substance may circulate even to portions besides the affected area and produce side effects. Therefore, during PDT, the administration of a photosensitive substance to only the affected area would be desirable.

### BRIEF SUMMARY

One object of the embodiments described herein is to provide an iontophoresis device for delivering a drug solution into part of an organism, such as a cancer site, for therapy or treatment using an endoscope or a laparoscope.

In one embodiment, a catheter-type iontophoresis device may include a working electrode assembly spaced apart from a non-working electrode assembly for administering a drug by iontophoresis, and a DC electric power source connected to the working electrode assembly and the non-working electrode assembly with opposite polarities. The catheter-type iontophoresis device may further include a rod-like member supporting the working electrode assembly and the non-working electrode assembly at its tip, and an endoscopic device for detachably supporting the rod-like member. The rod-like member may be detachably supported at a tip of a flexible cable supported by the endoscopic device.

In one embodiment, the drug may be a photosensitive material that is activated by absorbing light, and the endoscopic device may include an optical system for applying light near the working electrode assembly.

In one embodiment, the endoscopic device may further include an imaging system having an optical fiber for transmitting light to an inside of an organism and an optical fiber for transmitting reflected light to an outside of the organism. In such an embodiment, the optical system may include the optical fiber for transmitting light to the inside of the organism.

In yet another embodiment, the flexible cable may include an electric power source side working electrode terminal and an electric power source side non-working electrode terminal connected via wiring to the DC electric power source. The rod-like member may include a working electrode side contact and a non-working electrode side contact at a proximal end thereof, which are detachably connected to the electric power source side working electrode terminal and the electric power source side non-working electrode terminal, respectively. The working electrode side contact and the non-working electrode side contact may be connected to a working electrode and a non-working electrode of the working electrode assembly and the non-working electrode assembly, respectively.

In yet another embodiment, the endoscopic device may further include a controller disposed between each of the electric power source side working electrode terminal and the electric power source side non-working electrode terminal and the DC electric power source. The controller may be configured to adjust a value for a current of the DC electric power source and an energization time period.

In yet another embodiment, the working electrode assembly and the non-working electrode assembly may be disposed such that central axes thereof are parallel to each other.

In another embodiment, the working electrode assembly and the non-working electrode assembly may be disposed such that central axes thereof spread apart in a proximal direction.

In another embodiment, the working electrode assembly and the non-working electrode assembly may be disposed such that central axes thereof spread apart in a distal direction.

In still another embodiment, the working electrode assembly may include a working electrode connected to the DC electric power source having a same polarity as that of a charged ion of the drug; an electrolyte solution holding portion holding an electrolyte solution, the electrolyte solution holding portion disposed on a front surface of the working electrode; a second ion exchange membrane permitting passage of ions having a polarity opposite to that of the charged ion of the drug, the second ion exchange membrane disposed on a front surface of the electrolyte solution holding portion; a drug solution holding portion holding the drug, the drug solution holding portion disposed on a front surface of the second ion exchange membrane; and a first ion exchange membrane permitting passage of ions having the same polarity as that of the charged ion of the drug, the first ion exchange membrane disposed on a front surface of the drug solution holding portion. The non-working electrode assembly may include: a non-working electrode connected to the DC electric power source with a polarity opposite to that of the charged ion of the drug; a second electrolyte solution holding portion holding a second electrolyte solution, the second electrolyte solution holding portion disposed on a front surface of the non-working electrode; a third ion exchange membrane permitting passage of ions having a polarity opposite to that of the charged ion of the drug, the third ion exchange membrane disposed on a front surface of the second electrolyte solution holding portion; a third electrolyte solution holding portion holding a third electrolyte solution, the third electrolyte solution holding portion disposed on a front surface of the third ion exchange membrane; and a fourth ion exchange membrane permitting passage of ions having a polarity opposite to that of the charged ion of the drug, the fourth ion exchange membrane disposed on a front surface of the third electrolyte solution holding portion.

Each of the working electrode assembly and the non-working electrode assembly in the catheter-type iontophoresis device described herein may be arranged at the tip of the flexible cable of the endoscopic device. An anti-cancer agent may thereby be delivered to a pinpoint area, such as a cancer site, in, for example, a digestive organ. This may facilitate more efficient therapy with relatively few side effects. In another embodiment, immediately after PDT, each of the working electrode assembly and the non-working electrode assembly may be exchanged and an anti-cancer agent administered. As a result, therapy and prevention of recurrence can be simultaneously performed.

The term “front surface,” as used in the specification including the foregoing description, refers to the surface that is closer to a biological interface during use (e.g., mounting) of a device. The term “proximal,” as used in the specification, refers to a direction pointing away from the biological interface during use, and the term “distal,” as used in the specification, refers to a direction pointing towards the biological interface during use.

### BRIEF DESCRIPTION OF SEVERAL VIEWS OF THE DRAWINGS

In the drawings, identical reference numbers identify similar elements. The sizes and relative positions of elements in the drawings are not necessarily drawn to scale. For example, the shapes of various elements and angles are not drawn to scale, and some of these elements have been arbitrarily enlarged and positioned to improve drawing legibility. Further, the particular shapes of the elements as drawn are not intended to convey any information regarding the actual shape of the particular elements and have been solely selected for ease of recognition in the drawings.

FIG. 1 is a perspective view of a catheter-type iontophoresis device, according to one illustrated embodiment.

FIG. 2 is an enlarged cross-sectional view of a portion of a working electrode assembly and a non-working electrode assembly of the catheter-type iontophoresis device of FIG. 1.

FIG. 3 is a plan view of another example working electrode assembly and non-working electrode assembly, according to one illustrated embodiment.

FIG. 4 is a plan view of still another example working electrode assembly and non-working electrode assembly, according to one illustrated embodiment.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures and methods associated with catheters, ionotophoresis devices and medical procedures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic may be included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

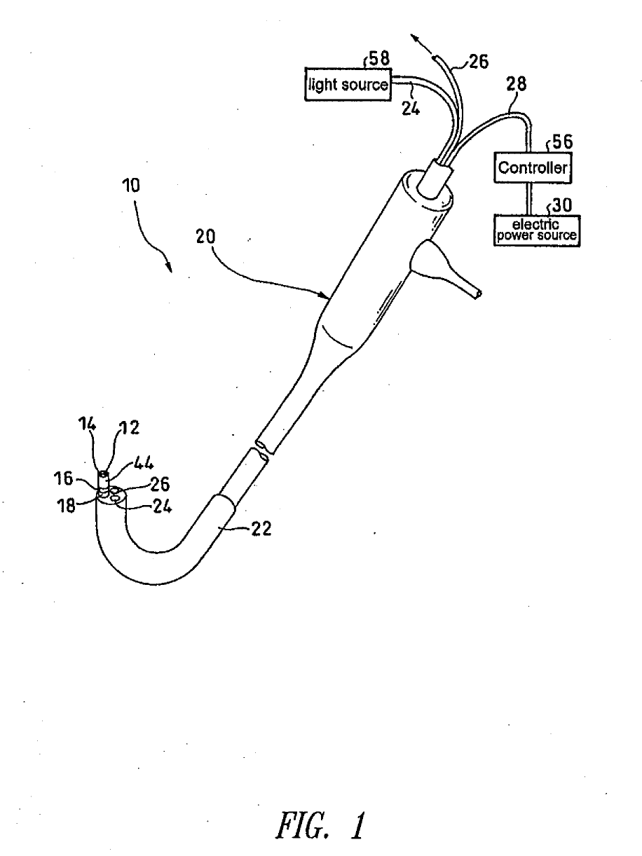

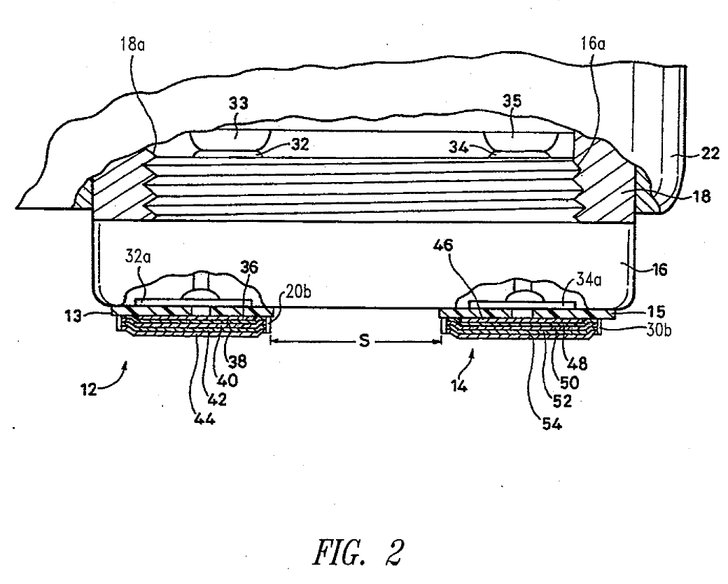

As shown in each of FIGS. 1 and 2, a catheter-type iontophoresis device 10 may include: a working electrode assembly 12 and a non-working electrode assembly 14 for administering a drug (e.g., an ionic drug); a rod-like member 16 for supporting the electrode assemblies 12, 14; and a DC electric power source 30 connected to the working electrode assembly 12 and the non-working electrode assembly 14 with different polarities.

Each of the working electrode assembly 12 and the non-working electrode assembly 14 may be attached to a tip of the rod-like member 16. The rod-like member 16 may further be detachably supported at a tip of a flexible cable 18. As a result, the working electrode assembly 12 and the non-working electrode assembly 14 may be integral with the rod-like member 16 and may be changed with replacement of the rod-like member 16.

The flexible cable 18 may in turn be supported by a flexible tube 22 of an endoscopic device 20 so that the cable 18 can freely curve. In one embodiment, the rod-like member 16 may be detachably attached to the tip of the flexible cable 18 projecting from the flexible tube 22.

The endoscopic device 20 may include an imaging system including an optical fiber 24 for transmitting generated light and an optical fiber 26 for transmitting reflected light, each optical fiber extending through the flexible tube 22. The optical fiber 24 may emit light from its tip to the inside of an organism. The optical fiber 26 may capture light reflected from the inside of the organism that was transmitted through the optical fiber 24 at, for example, an affected area in the organism and guide that reflected light to the outside of the organism. In one embodiment, white light may be directed from a light source 58 (e.g., a laser light source) through the optical fiber 24.

The working electrode assembly 12 and the non-working electrode assembly 14 may be connected to different polarities of the DC electric power source 30 via an electric power source circuit 28.

In one embodiment, a tip of the rod-like member 16 facing the flexible cable 18 may include a working electrode contact 32 connected to the working electrode assembly 12 and a non-working electrode contact 34 connected to the non-working electrode assembly 14.

The working electrode contact 32 and the non-working electrode contact 34 may be adapted to connect to an electric power source side working electrode terminal 33 and an electric power source side non-working electrode terminal 35 on the side of the flexible cable 18, respectively, when the rod-like member 16 is attached to the flexible cable 18.

The electric power source side working electrode terminal 33 and the electric power source side non-working electrode terminal 35 may be further connected to the DC electric power source 30 arranged further outside the endoscopic device 20 via the electric power source circuit 28.

In one embodiment, the rod-like member 16 may be a cylindrical member having the same diameter as that of the flexible cable 18. As shown in FIG. 2, the rod-like member 16 may be adapted to attach to the flexible cable 18 by threading a male screw portion 16a into a female screw portion 18a at the tip of the flexible cable 18. The rod-like member 16 may then be detached by rotating the male screw portion 16a in the opposite direction. Of course, other geometries and configurations are also possible.

FIG. 2 is an enlarged, cross-sectional view showing an arrangement in which the working electrode assembly 12 and the non-working electrode assembly 14 have parallel central axis lines.

In one embodiment, the working electrode assembly 12 may be formed by laminating a working electrode 36, an electrolyte solution holding portion 38, a second ion exchange membrane 40, a drug solution holding portion 42, and a first ion exchange membrane 44 in the above order from a side of the rod-like member 16. Although the working electrode assembly 12 may have any dimensions, in one embodiment, the assembly 12 forms a disk of about 2 to 6 mm in diameter.

The working electrode 36 may comprise a conductive paint applied to one surface of a base sheet 13 and blended with a nonmetal conductive filler, such as a carbon paste. In other embodiments, the working electrode 36 may comprise a copper plate or a metal thin film.

The electrolyte solution holding portion 38 may comprise, in one embodiment, an electrolytic paint applied to the working electrode 36. The electrolytic paint may comprise any paint containing an electrolyte, and, in one embodiment, an electrolyte that is oxidized or reduced more easily than water may be used. Examples of suitable electrolytes include: medical agents (e.g., ascorbic acid (vitamin C) and sodium ascorbate); and organic acids (e.g., lactic acid, oxalic acid, malic acid, succinic acid, and fumaric acid and/or salts thereof). The use of such electrolytes may suppress the generation of oxygen or hydrogen. In one embodiment, blending a plurality of electrolytes serving as a combination of buffer electrolyte solutions when dissolved in a solvent may suppress a change in pH during energization.

The electrolytic paint may be blended with a hydrophilic polymer, such as polyvinyl alcohol, polyacrylic acid, polyacrylamide, or polyethylene glycol in order to improve application and the film-forming property of the paint. The electrolytic paint may also be blended with a solvent, such as water, ethanol, or propanol, for adjusting the viscosity of the electrolytic paint. The electrolytic paint may also be blended with other components, such as a thickener, a thixotropic agent, a defoaming agent, a pigment, a flavor, and/or a coloring agent.

The second ion exchange membrane 40 may be formed by applying a second ion exchange paint to the electrolyte solution holding portion 38.

The second ion exchange paint may include an ion exchange resin into which an ion exchange group has been introduced, using, for example, as a counter ion, an ion having a polarity opposite to that of a drug ion in the drug solution holding portion 42. For example, if a drug whose drug component dissociates to positive drug ions is used in the drug solution holding portion 42, the second ion exchange paint may be blended with an anion exchange resin. On the other hand, if a drug whose drug component dissociates to negative drug ions is used, the second ion exchange paint may be blended with a cation exchange resin.

The drug solution holding portion 42 may comprise a drug paint applied to the second ion exchange membrane 40. In one embodiment, the drug paint may contain a drug (including a precursor for the drug) whose drug component dissociates to positive or negative ions (drug ions) as a result of, for example, dissolution into a solvent such as water. Examples of drugs whose drug components dissociate to positive ions include lidocaine hydrochloride and morphine hydrochloride, both as anesthetics. An example of a drug whose drug component dissociates to negative ions is ascorbic acid as a vitamin agent.

The first ion exchange membrane 44 may comprise a first ion exchange paint applied to the drug solution holding portion 42. In one embodiment, the first ion exchange paint may contain an ion exchange resin into which an ion exchange group is introduced using, for example, as a counter ion, an ion having the same polarity as that of the drug ion in the drug solution holding portion 42. Thus, if a drug whose drug component dissociates to positive drug ions is used in the drug solution holding portion 42, the paint may be blended with a cation exchange resin and vice versa.

The above-described ion exchange resins may be obtained by the following methods. In one embodiment, a cation exchange group (i.e., an exchange group using a cation as a counter ion), such as a sulfonic group, a carboxylic group, or a phosphoric group, may be introduced into a polymer having a three-dimensional network structure, such as a hydrocarbon-based resin (e.g., a polystyrene resin or an acrylic resin) or a fluorine-based resin having a perfluorocarbon skeleton. In another embodiment, an anion exchange group (i.e., an exchange group using an anion as a counter ion), such as a primary amino group, a secondary amino group, a tertiary amino group, a quaternary ammonium group, a pyridyl group, an imidazole group, a quaternary pyridinium group, or a quaternary imidazolium group, may be introduced into a polymer having a three-dimensional network structure, such as that used to form the cation exchange resin. Of course, other ion exchange resins may be used in other embodiments.

In one embodiment, the non-working electrode assembly 14 may be formed by laminating a non-working electrode 46, a second electrolyte solution holding portion 48, a third ion exchange membrane 50, a third electrolyte solution holding portion 52, and a fourth ion exchange membrane 54 in the above order on one side of a non-working base sheet 15. The non-working electrode assembly 14 may also form a disk similar in size and shape to the working electrode assembly 12.

The non-working electrode 46 may be formed similarly to the working electrode 36 of the working electrode assembly 12. In addition, the arrangement and composition of the second electrolyte solution holding portion 48 and the third electrolyte solution holding portion 52 may be the same as or similar to the electrolyte solution holding portion 38.

The third ion exchange membrane 50 may comprise an ion exchange paint applied to the second electrolyte solution holding portion 48. The ion exchange paint may be the same as or similar to the first ion exchange paint from which the first ion exchange membrane 44 is formed and may function as an ion exchange membrane in a manner similar to the first ion exchange membrane 44.

The fourth ion exchange membrane 54 may comprise the same ion exchange paint as that described above with respect to the second ion exchange membrane 40. The fourth ion exchange membrane 54 may function in a manner similar to the second ion exchange membrane 40.

A working electrode terminal plate 32a may be arranged on a side of the base sheet 13 opposite the working electrode 36, and conduction may be established between the working electrode terminal plate 32a and the working electrode 36 through a through-hole formed in the base sheet 13. The working electrode 36 may thus be connected to the working electrode contact 32 through the through-hole.

Similarly, a non-working electrode terminal plate 34a may be arranged on a side of the non-working base sheet 15 opposite the non-working electrode 46, and conduction may be established between the non-working electrode terminal plate 34a and the non-working electrode 46 through a through-hole formed in the non-working base sheet 15. The non-working electrode 46 may thus be connected to the non-working electrode contact 34 through the through-hole.

The first ion exchange membrane 44 and the fourth ion exchange membrane 54 at the tips of the working electrode assembly 12 and the non-working electrode assembly 14 may be exposed so as to be capable of contacting a biological interface of an organism.

In one embodiment, the DC electric power source 30 may comprise an AC to DC converter, and the electric power source circuit 28 between the DC electric power source 30 and the electric power source side working electrode terminal 33 and between the DC electric power source 30 and the electric power source side non-working electrode terminal 35 may include a controller 56 for adjusting a value for the current from the DC electric power source and/or an energization time period (corresponding to the administration time). As a result, each of the current value and the administration time may be adjusted within a certain range.

In the embodiment illustrated in FIG. 2, a spacing S (i.e., a predetermined amount of spacing) may be provided between the first ion exchange membrane 44 and the fourth ion exchange membrane 54 at the tips of the working electrode assembly 12 and the non-working electrode assembly 14, respectively, in order to prevent a current from directly flowing between the membranes upon energization. In one embodiment, the spacing S may have substantially the same dimension as a diameter of the first ion exchange membrane 44.

During therapy, the following procedure may be performed. White light may be applied through the optical fiber 24, serving as irradiation light. Reflected light (e.g., an image) may be transmitted to the outside by means of the optical fiber 26. An affected area may thereby be identified based on the reflected light. The working electrode assembly 12 and/or the non-working electrode assembly 14 may then be pressed against the affected area. In one embodiment, the lighting may be turned off during iontophoresis.

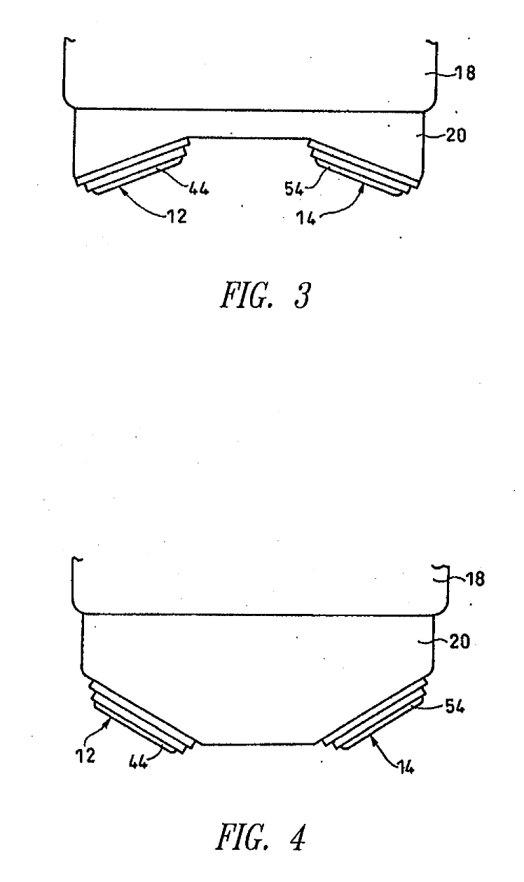

In the above embodiment, the working electrode assembly 12 and the non-working electrode assembly 14 are attached such that their central axes are parallel with each other. However, other embodiments are also possible, in which the central axes spread apart in the proximal or distal directions. For example, as shown in FIG. 3, the working electrode assembly 12 and the non-working electrode assembly 14 may be arranged such that their central axes intersect in a direction extending from the tip (i.e., spread apart in a proximal direction). In one embodiment, the axes may form an angle of approximately 60° between them. Alternatively, as shown in FIG. 4, the working electrode assembly 12 and the non-working electrode assembly 14 may also be arranged such that their central axes spread apart in a distal direction.

In each embodiment, the working electrode assembly 12 and the non-working electrode assembly 14 may be arranged at a tip of the flexible cable 18 of the endoscopic device 20 with a spacing S between them. Therefore, for example, when a drug solution is delivered to a cancer site of a digestive organ, a doctor may grip the endoscopic device 20 to bring the first ion exchange membrane 44 at a tip of the working electrode assembly 12 at a tip of the flexible cable 18 into close contact with the cancer site and, at the same time, to bring the fourth ion exchange membrane 54 at a tip of the non-working electrode assembly 14 into close contact with a mucosa or other biological interface near the cancer site for energization. Thus, a drug solution may be delivered to a target site on a pinpoint basis.

In one embodiment, the working electrode assembly 12 and the non-working electrode assembly 14 can be detached together with the rod-like member 16 from the flexible cable 18, so that a different drug solution may be delivered.

The catheter-type iontophoresis device 10 may be used for therapy on the inside of an organism using PDT, e.g., as an anti-cancer remedy, by: delivering a photosensitive substance to a cancer cell; and irradiating the substance with light to cause the substance to absorb the light. For example, the device 10 can be used to treat a superficial esophageal cancer, a superficial gastric cancer, or a cervical cancer. In addition, the device 10 may be used for other types of therapy within an organism, such as treating a gastric ulcer or colitis.

When using the device 10 in PDT, the drug solution holding portion 42 of the working electrode assembly 12 may hold a photosensitive substance, and light having a wavelength appropriate for absorption by the photosensitive substance, such as ultraviolet light, may be supplied from a light source 58 via the optical fiber 24 to irradiate an affected area under control of the controller 56. In one embodiment, a light source emitting the light for PDT may be arranged separately from a source of white light, and the white light and the PDT light may be selected by switching the light input to the optical fiber 24 (not shown). In another embodiment, a filter passing only light having the correct PDT wavelength may be used to selectively filter the white light instead, without the use of a new light source.

### DESCRIPTION OF REFERENCE NUMERALS

10 CATHETER-TYPE IONTOPHORESIS DEVICE
12 WORKING ELECTRODE ASSEMBLY
14 NON-WORKING ELECTRODE ASSEMBLY
16 ROD-LIKE MEMBER
18 FLEXIBLE CABLE
20 ENDOSCOPIC DEVICE
22 FLEXIBLE TUBE
24 OPTICAL FIBER FOR IRRADIATION
26 OPTICAL FIBER FOR REFLECTED LIGHT
28 ELECTRIC POWER SOURCE CIRCUIT
30 DC ELECTRIC POWER SOURCE
32 WORKING ELECTRODE CONTACT
33 ELECTRIC POWER SOURCE SIDE WORKING ELECTRODE TERMINAL
34 NON-WORKING ELECTRODE CONTACT
35 ELECTRIC POWER SOURCE SIDE NON-WORKING ELECTRODE TERMINAL
36 WORKING ELECTRODE
38 ELECTROLYTE SOLUTION HOLDING PORTION
40 SECOND ION EXCHANGE MEMBRANE
42 DRUG SOLUTION HOLDING PORTION
44 FIRST ION EXCHANGE MEMBRANE
46 NON-WORKING ELECTRODE
48 SECOND ELECTROLYTE SOLUTION HOLDING PORTION
50 THIRD ION EXCHANGE MEMBRANE
52 THIRD ELECTROLYTE SOLUTION HOLDING PORTION
54 FOURTH ION EXCHANGE MEMBRANE
56 CONTROLLER
58 LIGHT SOURCE

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

The various embodiments described above can be combined to provide further embodiments. From the foregoing it will be appreciated that, although specific embodiments have been described herein for purposes of illustration, various modifications may be made without deviating from the spirit and scope of the teachings. Accordingly, the claims are not limited by the disclosed embodiments.

We claim:

1. A catheter-type iontophoresis device, comprising:
a working electrode assembly spaced apart from a non-working electrode assembly for administering a drug by iontophoresis;
a DC electric power source connected to the working electrode assembly and the non-working electrode assembly with opposite polarities:
a rod-like member supporting the working electrode assembly and the non-working electrode assembly at a tip of the rod-like member; and
an endoscopic device detachably supporting the rod-like member,
the rod-like member detachably supported at a tip of a flexible cable supported by the endoscopic device.

2. The catheter-type iontophoresis device of claim 1, wherein the drug comprises a photosensitive material that is activated by absorbing light, and the endoscopic device includes an optical system for applying light near the working electrode assembly.

3. The catheter-type iontophoresis device of claim 2, wherein:
the endoscopethe endoscopic device further includes an imaging system having an optical fiber for transmitting light to an inside of an organism and an optical fiber for transmitting reflected light to an outside of the organism; and
the optical system includes the optical fiber for transmitting light to the inside of the organism.

4. The catheter-type iontophoresis device of claim 1 wherein:
the flexible cable includes an electric power source side working electrode terminal and an electric power source side non-working electrode terminal connected via wiring to the DC electric power source;
the rod-like member includes a working electrode side contact and a non-working electrode side contact at a proximal end thereof, which are detachably connected to the electric power source side working electrode terminal and the electric power source side non-working electrode terminal, respectively; and
the working electrode side contact and the non-working electrode side contact are connected to a working electrode and a non-working electrode the working electrode assembly and the non-working electrode assembly, respectively.

5. The catheter-type iontophoresis device of claim 4, wherein the endoscopic device further comprises a controller configured to adjust a value for a current of the DC electric power source and an energization time period, the controller disposed between each of the electric power source side working electrode terminal and the electric power source side non-working electrode terminal and the DC electric power source.

6. The catheter-type iontophoresis device of claim 1, wherein the working electrode assembly and the non-working electrode assembly are arranged such that central axes thereof are parallel to each other.

7. The catheter-type iontophoresis device of claim 1, wherein the working electrode assembly and the non-working electrode assembly are arranged such that central axes thereof spread apart in a proximal direction.

8. The catheter-type iontophoresis device of claim 1, wherein the working electrode assembly and the non-working electrode assembly are arranged such that central axes thereof spread apart in a distal direction.

9. The catheter-type iontophoresis device of claim 1, wherein the working electrode assembly comprises:
the a working electrode connected to the DC electric power source having a same polarity as that of a charged ion of the drug;
an electrolyte solution holding portion holding an electrolyte solution, the electrolyte solution holding portion disposed on a front surface of the working electrode;
a second ion exchange membrane passage of ions having a polarity opposite to that of the charged ion of the drug, the second ion exchange membrane disposed on a front surface of the electrolyte solution holding portion;
a drug solution holding portion holding the drug, the drug solution holding portion disposed on a front surface of the second ion exchange membrane; and
a first ion exchange membrane permitting of ions having the same polarity as that of the charged ion of the drug, the first ion exchange membrane disposed on a front surface of the drug solution holding portion; and
wherein the non-working electrode assembly comprises:
the a non-working electrode connected to the DC electric power source with the polarity opposite to that of the charged ion of the drug;
a second electrolyte solution holding portion holding a second electrolyte solution, the second electrolyte solution holding portion disposed on a front surface of the non-working electrode;
a third ion exchange membrane permitting passage of ions having the polarity opposite to that of the charged ion of the drug, the third ion exchange membrane disposed on a front surface of the second electrolyte solution holding portion;
a third electrolyte solution holding portion holding a third electrolyte solution, the third electrolyte solution holding portion disposed on a front surface of the third ion exchange membrane; and
a fourth ion exchange membrane permitting passage of ions having the polarity opposite to that of the charged ion of the drug, the fourth ion exchange membrane disposed on a front surface of the third electrolyte solution holding portion.
